(19) United States
(12) Patent Application Publication
(10) Pub. No.: US 2006/0009702 A1
(43) Pub. Date: Jan. 12, 2006

(54) User support apparatus

(75) Inventors: Hidekazu Iwaki, Hachioji-shi (JP); Takashi Miyoshi, Atsugi-shi (JP); Akio Kosaka, Hachioji-shi (JP)

Correspondence Address: FRISHAUF, HOLTZ, GOODMAN & CHICK, PC, 220 5TH AVE FL 16, NEW YORK, NY, 10001-7708, US

(73) Assignee: Olympus Corporation, Tokyo (JP)

(21) Appl. No.: 11/115,827

(22) Filed: Apr. 27, 2005

(30) Foreign Application Priority Data
Apr. 30, 2004 (JP) 2004-136205

(51) Int. Cl. A61B 5/04

(52) U.S. Cl. 600/520

(57) ABSTRACT

A user support apparatus comprises a user information acquisition unit configured to obtain information regarding a user, a user state estimation unit configured to judge at least one of user's position information, posture information, physical sate, and mental state as a user state based on the information obtained by the user information acquisition unit, and a user support unit configured to support at least one of user's action, memory and thinking based on the user state estimated by the user state estimation unit.

### CROSS-REFERENCE TO RELATED APPLICATIONS

This application is based upon and claims the benefit of priority from prior Japanese Patent Application No. 2004-136205, filed Apr. 30, 2004, the entire contents of which are incorporated herein by reference.

### BACKGROUND OF THE INVENTION

1. Field of the Invention

The present invention relates to a user support apparatus which supports user's action, memory or thinking.

2. Description of the Related Art

Jpn. Pat. Appln. KOKAI Publication No. 2001-56225 proposes an agent system which performs communication with a user by judging a vehicle situation. According to this agent system, an action that can be processed by an agent is proposed: for example, “do you need restaurant directions?” when user's stomach sound is detected at lunchtime. For an agent to emerge in the vehicle, the user can select its appearance or voice to taste.

### BRIEF SUMMARY OF THE INVENTION

According to a first aspect of the present invention, there is provided a user support apparatus comprising:
a user information acquisition unit configured to obtain information regarding a user;
a user state estimation unit configured to estimate at least one of user's position information, posture information, physical sate, and mental state as a user state based on the information obtained by the user information acquisition unit; and
a user support unit configured to support at least one of user's action, memory and thinking based on the user state estimated by the user state estimation unit.

According to a second aspect of the present invention, there is provided a user support apparatus comprising:
a user sate estimation unit including at least two of a user external information acquisition unit configured to obtain user external information which is information sensed by a user, a user internal information acquisition unit configured to obtain user internal information which is user's own information, and an environmental information acquisition unit configured to obtain environmental information around the user, and configured to estimate a user state containing at least one of user's position information, posture information, physical state and mental state based on the pieces of information obtained by the acquisition units; and
a user support unit configured to support at least one of user's action, memory and thinking based on the user state estimated by the user state estimation unit.

According to a third aspect of the present invention, there is provided a user support apparatus comprising:
user information acquisition means for acquiring information regarding a user;
user state estimation means for estimating at least one of user's position information, posture information, physical sate, and mental state as a user state based on the information obtained by the user information obtaining means; and
user support means for supporting at least one of user's action, memory and thinking based on the user state estimated by the user state estimation means.

According to a fourth aspect of the present invention, there is provided a user support apparatus comprising:
user sate estimation means including at least two of user external information obtaining means for acquiring user external information which is information sensed by a user, user internal information acquisition means for obtaining user internal information which is user's own information, and environmental information acquisition means for obtaining environmental information around the user, and for estimating a user state containing at least one of user's position information, posture information, physical state and mental state based on the pieces of information obtained by the acquisition means; and
user support means for supporting at least one of user's action, memory and thinking based on the user state estimated by the user state estimation means.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a view showing a configuration of a user support apparatus according to a first embodiment of the present invention;

FIG. 2A is a perspective view a spectacles type stereo camera as an example of a user external information acquisition unit including a user support unit;

FIG. 2B is a back view showing the spectacles type stereo camera;

FIG. 3 is a perspective view showing a pendant-type stereo camera as another example of a user external information acquisition unit including a user support unit;

FIG. 4 is a view showing a configuration until stimulus information is presented to the user support unit as a specific example of the first embodiment;

FIG. 5 is a view showing a configuration until danger information is presented to the user support unit as a specific example of the first embodiment;

FIG. 6 is a view showing a configuration of a user support apparatus according to a second embodiment of the present invention;

FIG. 7 is a view showing a configuration until danger information is presented to a user support unit as a specific example of the second embodiment;

FIG. 8 is a flowchart showing an operation of generating track prediction data and of transmitting information to a user state estimation unit in an environmental information acquisition unit;

FIG. 9 is a flowchart showing user state estimation unit's operation of generating subjective space danger degree data from left and right camera image data from a user external information acquisition unit, and physical recognition data and the track prediction data from the environmental information acquisition unit;

FIG. 10 is a view showing a configuration until information based on user's taste to the user support unit as a specific example of the second embodiment;

FIG. 11 is a flowchart showing an operation of generating support data in the user state estimation unit;

FIG. 12A is a view showing a specific information presentation example when a car comes from the right in a user support apparatus according to a third embodiment of the present invention;

FIG. 12B is a view showing a specific information presentation example when a free taxi comes from the right in the user support apparatus of the third embodiment;

FIG. 13A is a view showing a specific information presentation example when an unknown human follows user's back for a predetermined time or a predetermined distance in the user support apparatus of the third embodiment;

FIG. 13B is a view showing a specific information presentation example when an unknown motorcycle follows user's back for a predetermined time or a predetermined distance in the user support apparatus of the third embodiment;

FIG. 13C is a view showing a specific information presentation example when a known human follows user's back for a predetermined time or a predetermined distance in the user support apparatus of the third embodiment;

FIG. 13D is a view showing another specific information presentation example when a known human follows user's back for a predetermined time or a predetermined distance in the user support apparatus of the third embodiment;

FIG. 14 is a view showing a specific information presentation example when a user is alerted in the user support apparatus of the third embodiment;

FIG. 15A is a view showing a specific information presentation example when navigation is carried out in the user support apparatus of the third embodiment;

FIG. 15B is a view showing another specific information presentation example when the navigation is carried out in the user support apparatus of the third embodiment;

FIG. 15C is a view showing yet another specific information presentation example when the navigation is carried out in the user support apparatus of the third embodiment; and

FIG. 16 is a view showing a specific information presentation example when a known human is identified in the user support apparatus of the third embodiment.

### DETAILED DESCRIPTION OF THE INVENTION

Hereinafter, the preferred embodiments of the present invention will be described with reference to the accompanying drawings.

### First Embodiment

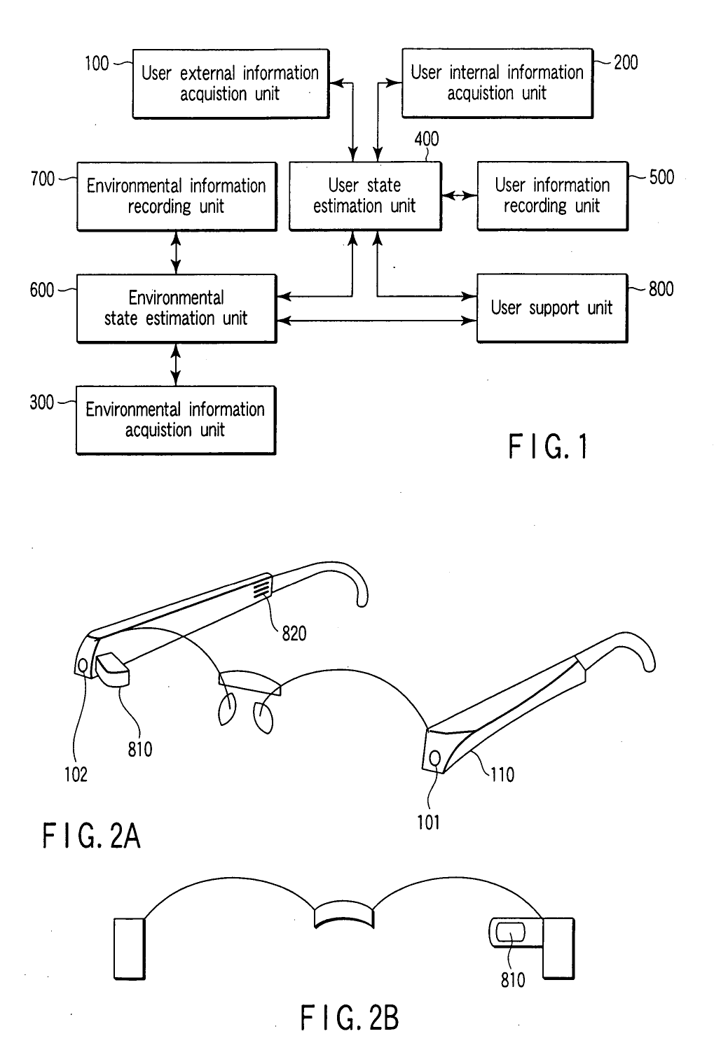

Referring to FIG. 1, a user support apparatus according to a first embodiment of the present invention comprises a user external information acquisition unit 100, a user internal information acquisition unit 200, an environmental information acquisition unit 300, a user state estimation unit 400, a user information recording unit 500, an environmental state estimation unit 600, an environmental information recording unit 700, and a user support unit 800.

The user external information acquisition unit 100 obtains user's external information, and the user internal information acquisition unit 200 obtains user's internal information. The environmental information acquisition unit 300 obtains information of an environment in which the user is present. It is to be noted that the user support apparatus of the embodiment does not always have all of the three information acquisition units. The apparatus needs to have at least two information acquisition units.

The user state estimation unit 400 estimates a state of the user based on the user's external and internal information obtained by the user external and internal information acquisition units 100 and 200. The user information recording unit 500 records the information estimated by the user state estimation unit 400 or the pieces of information themselves obtained by the user external and internal information acquisition units 100 and 200. The environmental state estimation unit 600 estimates a state of an environment based on the environmental information obtained by the environmental information acquisition unit 300. The environmental information recording unit 700 records the information estimated by the environmental state estimation unit 600 or the information itself obtained by the environmental information acquisition unit 300. The user support unit 800 supports the user by presenting the pieces of information recorded by the user information recording unit 500 and the environmental information recording unit 700 or outputs from the user state estimation unit 400 and the environmental state estimation unit 600 to the user.

Hereinafter, each unit of the user support apparatus will be described in detail.

The user external information acquisition unit 100 has a support area which is a range that the user can sense at least by some of five senses, i.e., an area that the user senses, and senses information (user external information) of the support area as needed. For example, the user external information obtained by the user external information acquisition unit 100 contains at least one of an image, a voice (or sound), an odor (direction of generation source), an air temperature, a humidity, components in air, an atmospheric pressure, brightness, an ultraviolet ray amount, an electric wave, a magnetic field, a ground temperature, IC tag information, a distance, and a wind direction. A sensor as the user external information acquisition unit 100 for acquisition such user external information is mounted to the user himself or herself. Needless to say, we here describe the simplest sensor placement configuration for the user, it is possible to use a sensor not directly mounted to the user: for example, use of a sensor fixed to a robot which moves together with the user, or switched use of a plurality of fixed sensors together with a movement of the user. In this case, information detected by the sensor only needs to be used after an attribute is added to indicate that it is also a content of user external information.

Additionally, for example, the user external information acquisition unit 100 senses an image of the area which the user cannot actually see but from which the user can hear a sound (e.g., behind the user). The user external information acquisition unit 100 may sense user's position and/or posture information by using at least one of a one or two-dimensional range sensor, a gravitational direction sensor, an acceleration sensor, an angular acceleration sensor, a gyrosensor, position and posture estimation information based on sensing of a mark fixed to the outside, and a GPS signal. The user external information acquisition unit 100 may further include an interface for receiving user's input. Accordingly, the user can supply comments on experiences or special notes.

Description of the sensors for sensing the information will be omitted as technologies therefor have been available. The pieces of sensed user external information are transmitted to the user state estimation unit 400 as needed.

The user internal information acquisition unit 200 senses information (user internal information) which becomes a criterion for user's physical condition, mental state, degree of excitement, or the like as needed. For example, the user internal information obtained by the user internal information acquisition unit 200 contains at least one of user's perspiration amount, electric skin potential response or level, eye movement, electromyogram, electroencephalogram, brain magnetic field, vital sign (blood pressure, pulsation, aspiration, or body temperature), facial expression, face color, voiceprint, body shakes, body movement, blood sugar level, body fat, and blood flow. A sensor as the user internal information acquisition unit 200 for detecting such user internal information is mounted to the user himself. Needless to say, as in the case of the user external information acquisition unit 100, the user internal information acquisition unit 200 can use a sensor not mounted to the user. Description of the sensors for sensing such information will be omitted as technologies therefor have been already known and available. The user internal information may a result of collecting saliva, breath, excrement, sweat, or the like together with a time stamp and analyzing a component off-line, or an analyzing result from transparency or color by an image. The sensed pieces of user internal information are transmitted to the user state estimation unit 400 as needed.

The environmental information acquisition unit 300 senses information in a predetermined area (environmental information) by a sensor fixed to a living space in which a group of users can be present as needed. Here, for example, the living space includes spaces in a room, a train, an elevator, a park, an airport, a hall, an urban area, a station, a highway, a vehicle, a building, and the like. For example, the environmental information obtained by the environmental information acquisition unit 300 contains at least one of an image, a voice (or sound), an odor (direction of generation source), an atmospheric pressure, a humidity, components in air, an atmospheric pressure, brightness, an ultraviolet ray amount, an electric wave, a magnetic field, a ground temperature, IC tag information, a distance, and a wind direction. The environmental information acquisition unit 300 may sense its own position and/or posture information by using at least one of a one or two-dimensional range sensor, a gravitational direction sensor, an accelerometer, an angular accelerometer, a gyrosensor, and a GPS signal. Many sensors for sensing the information have been commercially available, and thus description thereof will be omitted. The sensed pieces of environmental information are transmitted to the environmental state estimation unit 600 as needed.

The user state estimation unit 400 estimates user's current or future state based on the user external and internal information sent from the user external and internal information acquisition unit 100 and 200, and past user information recorded in the user information recording unit 500. For example, the user state estimation unit 400 recognizes at least one of an interest area (pointing direction, user's visual field, visual line or body direction) which the user pays attention to, user's degrees of stress, excitement, impression, fatigue, attention, fear, concentration, interest, jubilation, drowsiness, excretion wish, appetite, and states of physical conditions and activities. It also decides a current action (speaking, moving, exercising, studying, working, sleeping, meditating, or resting). Then, the user state estimation unit 400 predicts user's future action from a result of the estimation, estimates information needed by the user, and transmits estimated contents to the user support unit 800. Here, within a support range of the environmental information acquisition unit 300, user's state information seen from a surrounding area may be obtained from the environmental state estimation unit 600, and used as supplementary information for estimating user's state. At least one of user's position or posture information, a point of attention, conversation contents, a conversation opponent, and conversation opponent's face color, vice tone, or visual line. A specific example will be described later. Further, the user state estimation unit 400 transmits the user external information, the user internal information, and the user state information together with a time stamp, or position information or the like to the user information recording unit 500.

The user information recording unit 500 records the information sent from the user state estimation unit 400 together with the time stamp. The user information recording unit 500 retrieves past information in response to an inquiry from the user state estimation unit 400, and transmits its result to the user state estimation unit 400. These pieces of information may be weighted by an interest area (pointing direction, user's visual field, visual line or body direction) which the user pays attention to, user's stress, degrees of excitement, impression, fatigue, attention, fear, concentration, interest, jubilation, drowsiness, excretion wish, appetite, states of physical conditions and activities, and a current action (speaking, moving, exercising, studying, working, sleeping, meditating, or resting), and organized.

The environmental state estimation unit 600 estimates an environmental state based on the environmental information sent from the environmental information acquisition unit 300 and the past environmental information recorded in the environmental information recording unit 700. For example, the environmental state estimation unit 600 recognizes at least one of operation history of the user group; a movement of an object group; an operation of a vehicle (automobile), an elevator, a gate, a door or the like; and weather. Then, the environmental state estimation unit 600 estimates the degree of danger around the user group present within the support range or information useful to the user, and transmits it to the user state estimation unit 400. Here, user state information of each user may be obtained from the user state estimation unit 400 present within the support range to be used for judging the environmental state. As the useful information sent to the user, static information which is unchanged information to a certain extent such as a map or a store recorded in the environmental information recording unit 700 may be referred to. A specific example will be described later. Further, the environmental state estimation unit 600 transmits the environmental information and the environmental state information together with the time stamp, the position information or the like to the environmental information recording unit 700. It is to be noted that the environmental state estimation unit 600 may be included in the environmental information acquisition unit 300.

The environmental information recording unit 700 records the information sent from the environmental state estimation unit 600 together with the time stamp, and also the static information such as a map or a store input from the external input. The environmental information recording unit 700 retrieves the past information or the static information in response to an inquiry from the environmental state estimation unit 600, and transmits its result to the environmental state estimation unit 600. These pieces of information may be weighted by an operation of the user group; a movement of the object group; an operation of the vehicle, the elevator, the gate or the door; and weather, and organized.

The user support unit 800 presents support information to the user by using at least one of a sound, an image, an odor, a tactile sense, and vibration based on the information sent from the user state estimation unit 400 and the environmental state estimation unit 600. This user support unit 800 may be included in the user external information acquisition unit 100, the user internal information acquisition unit 200, or the user state estimation unit 400.

For example, as shown in FIGS. 2A and 2B, the user support unit 800 can be included in the eyeglass type stereo camera 110 as the user external information acquisition unit 100. That is, the eyeglass type stereo camera 110 incorporates a left camera 101 at a left end of a spectacles type frame and a right camera 102 at a right end. These two cameras 101, 102 constitute the stereo camera. In the temple portion of the eyeglass type frame, a stereo microphone, a gyrosensor, an acceleration sensor, a GPS sensor, a passometer or the like (not shown) may be incorporated. As the user support unit 800, a microdisplay (head-mounted display) 810 is incorporated in the front of the eyeglass type frame within user's visual field, and a speaker 820 is incorporated in the frame near user's ear. In other words, various pieces of information can be supplied to the user by visually presenting information on the microdisplay 810, and information as a sound can be presented to the user through the speaker 820. It is to be noted that the microdisplay 820 has a function of displaying an image captured by the left or right camera 101 or 102, and information can be superimposed by superimposing character information, icon information or visual information on the captured image and adding it to a target object detected within camera's visual field. Moreover, the display is a see-through (half-transparent) type, and information may be presented to be superimposed on a real world seen-through.

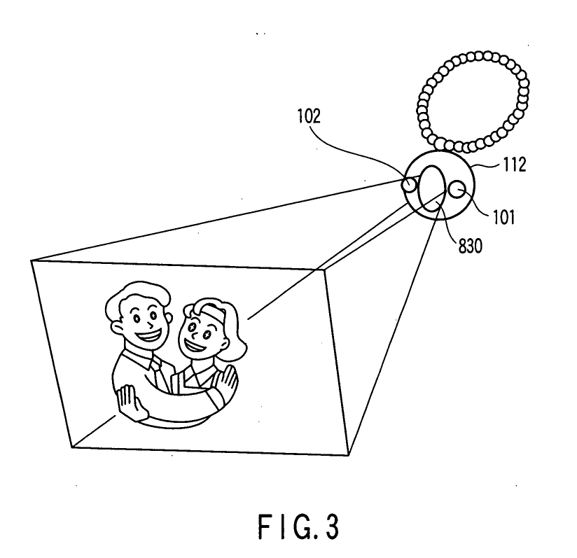

Furthermore, as shown in FIG. 3, the user support unit 800 may be included in a pendant type stereo camera 112 as the user external information acquisition unit 100. That is, this pendant type stereo camera 112 incorporates left and right cameras 101, 102. The two cameras 101, 102 constitute the stereo camera. The pendant type stereo camera 112 incorporates a gyrosensor, an acceleration sensor, a passometer, a GPS sensor, a stereo microphone or the like. The pendant type stereo camera 112 further incorporates a microprojector 830 as the user support unit 800. This microprojector 830 presents information by, e.g., projecting a light on user's palm. Needless to say, each of the aforementioned portions may be software or exclusively designed hardware operated on a personal computer, a wearable computer, or a general-purpose computer such as a grid computer, or through networks of a plurality of general-purpose computers.

Now, to assist the understanding of the embodiment, the operation of the user support apparatus of the embodiment will be described by assuming a specific scene.

(Scene 1)

An example of giving a stimulus to the user based on information from the environmental estimation unit 600 will be described.

When the user stops before a sculpture at a certain street corner to watch it, the user external information acquisition unit 100 captures the scene by the stereo camera, and a group of captured images are sent to the user state estimation unit 400. The user state estimation unit 400 recognizes shaking motion of the stereo camera from the sent images, determines user's fixed gaze, and measures a degree of interest based on the gazing time. If the degree of interest exceeds a certain threshold value, estimating that the user takes interest in the sculpture, sculpture related information is generated as user support information. Specifically, a label of the sculpture is read from the group of photographed images to obtain author's name. Further, the sculpture is three-dimensionally reconstructed from a stereo image obtained from the stereo camera to obtain a three-dimensional range map. Then, based on the three-dimensional range map or the author's name, an inquiry is made to the environmental state estimation unit 600 for detailed information of the work, a sculpture of a similar shape, and works of the same author. The environmental state estimation unit 600 sends information retrieved from the environmental information recording unit 700 to the user state estimation unit 400. The user state estimation unit 400 presents the detailed information of the work and the information regarding the sculpture of the similar shape or works of the same author through the user support unit 800 to the user in accordance with the received information. Further, the sculpture image, the author information, similar work information, a current position, a time, and the three-dimensional information are transmitted together with a degree of interest in the presentation to the user information recording unit 500, and recorded. The user information recording unit 500 sets a compression rate based on the degree of interest, and records the information as user's unique taste information by adding a label if the degree of interest exceeds a certain threshold value.

The aforementioned operation can stimulate user's intellectual curiosity, and support the user.

In this example, the user external information acquisition unit 100 comprises a stereo camera, the user state estimation unit 400 comprises a user gazing degree determination device, a user interest degree determination device, and a three-dimensional reconstruction device, the environmental state estimation unit 600 comprises a sculpture three-dimensional retrieval engine, the environmental information recording unit 700 comprises a sculpture three-dimensional database, and the user information recording unit 500 comprises an information compressing unit.

(Scene 2)

An example of detecting a user state and giving guidance or navigation of a store or the like will be described.

The user internal information acquisition unit 200 always monitors a blood sugar level of the user to transmit it to the user state estimation unit 400. The user state estimation unit 400 monitors user's hunger from a change in the blood sugar level. When the hunger (appetite) is detected, a menu candidate for meals is generated by referring to a keyword or the like which appears in past meal contents, an exercise amount, taste or conversation from the user information recording unit 500. Next, an inquiry is made to the environmental state estimation unit 600 to recognize a current position from the GPS information of the user external information acquisition unit 100, and a nearby store restaurant candidate where menu candidate will be offered is presented to the user. When the user selects a menu and/or a store, the user state estimation unit 400 makes an inquiry to the environmental state estimation unit 600 about an optimal route to the selected store, presents the route to the store to the user support unit 800, and navigates the route as needed. The information regarding the selected menu and the store is recorded as taste information in the user information recording unit 500 together with a degree of excitement estimated by the user state estimation unit 400 using a conversation during a meal obtained from the user external information estimation unit 100 or a heart rate obtained from the user internal information acquisition unit 200.

The aforementioned operation enables presentation of an optimal meal when hungry, thereby supporting the user.

In this example, the user external information acquisition unit 100 comprises a microphone and a GPS signal receiver, the user internal information acquisition unit 200 comprises a blood sugar level monitor and a heart rate monitor, the user state estimation unit 400 comprises a hunger determination device, a preferred menu candidate generator, a store information referring device, a conversation understanding device, and an excitement degree determination device, the environmental state estimation unit 600 comprises a store retrieval engine, the environmental information recording unit 700 comprises a store database, and the user information recording unit 500 comprises meal history and exercise history databases.

(Scene 3)

An example of recognizing a degree of user's boredom to present stimulus information will be described.

The user state estimation unit 400 recognizes a state of many yawns, or a state of no action, i.e., a bored state, based on a stereo image from the user external information acquisition unit 100. In such a state, the user state estimation unit 400 recognizes a current position from the GPS signal from the user external information acquisition unit 100, and makes an inquiry to the environmental state estimation unit 600 to present, based on user's taste information recorded in the user information recording unit 500, information such as film showing information or new boutique information around the current position, relaxation spot information such as massages or hot springs, new magazine or book information, or demonstration sales information of latest personal computers or digital cameras to the user support unit 800. Among such pieces of information, for example, if the massage is selected, a taste degree of massages in the user information recording unit 500 is increased, an inquiry is made to the environmental state estimation unit 600 about an optimal route to the selected massage parlor. The route to the parlor is presented to the user support unit 800, and navigated as needed. Subsequently, based on the heart rate and a blood pressure obtained by the user internal information acquisition unit 200, the user state estimation unit 400 judges an effect of the massage, and records its evaluation value together with a place of the parlor in the user information recording unit 500.

The aforementioned operation enables effective spending of a bored time, thereby supporting the user.

In this example, the user external information acquisition unit 100 comprises a stereo camera and a GPS receiver, the user internal information acquisition unit 200 comprises a heart rate monitor and a blood pressure monitor, the user state estimation unit 400 comprises a three-dimensional reconstruction device, a yawn detector, and a boredom degree determination device, the environmental state estimation unit 600 comprises an amusement retrieval engine, and the environmental information recording unit 700 comprises an amusement database.

(Scene 4)

An example of an operation of estimating a user state to increase concentration will be described.

Based on the stereo image from the user external information acquisition unit 100, the user state estimation unit 400 recognizes a state of writing something by watching a text, i.e., studying. In such a case, the user state estimation unit 400 plays favorite music recorded in the user information recording unit 500 or music to facilitate concentration through the speaker of the user support unit 800, or generate a fragrance to increase concentration. As a result, a change in an electroencephalogram is measured by the user internal information acquisition unit 200, an effect of the music or the fragrance is estimated by the user state estimation unit 400, and an estimating result is recorded with information to specify each music or fragrance in the user information recording unit 500. In this case, in place of the brain wave, the concentration may be determined based on a writing speed, a responding speed, or a visual point track. Additionally, diversion may be urged if the concentration is not increased so much.

The aforementioned operation enables work while high concentration is maintained, thereby supporting the user.

In this example, the user external information acquisition unit 100 comprises a stereo camera, the user internal information acquisition unit 200 comprises a brain wave monitor, the user state estimation unit 400 comprises a three-dimensional reconstruction device, a reference object recognition device, a writing operation detector, and a concentration determination device, and the user support unit 800 comprises a speaker and a fragrance generator.

(Scene 5)

An example of estimating an environmental state to call attention will be described.

The user state estimation unit 400 can recognize approaching of an object from the front at a certain relative speed based on the stereo image from the user external information unit 100. The environmental state estimation unit 600 can recognize approaching of an object at a blind angle of the user at a certain relative speed based on the stereo image obtained by the environmental information acquisition unit 300. When such recognition is made, the environmental state estimation unit 600 first makes an inquiry to the user sate estimation unit 400 about the target object, and a relation with the user himself is estimated by the user state estimation unit 400 of the user. For this blind angle, the user state estimation unit 400 recognizes a range supported by the user external information acquisition unit 100, and sets a portion other than the range as a blind angle. The relation includes a relation with a past encountered object recorded in the user information recording unit 500. When there is no relation at all, but there is a danger of collision, or when a similar state is retrieved from past danger states recorded in the user information recording unit 500 (e.g., collision with an automobile), a danger of collision is presented to the user support unit 800. Additionally, this situation (place, time, target object, or collision possibility) is recorded as a danger state together with a fear degree in the user information recording unit 500.

The aforementioned operation enables predetection of a danger, thereby supporting the user.

In this example, the user external information acquisition unit 100 comprises a stereo camera, the user state estimation unit 400 comprises a front object recognition device, a user blind angle recognition device, a three-dimensional reconstruction device, and a relation retrieval engine, the environmental information acquisition device 300 comprises a stereo camera, the environmental state estimation unit 600 comprises a three-dimensional reconstruction device, an object recognition device and a danger state retrieval engine, the environmental information recording unit 700 comprises a danger state database, and the user information recording unit 500 comprises an object relation database.

Hereinafter, by limiting functions for simplicity, a specific configuration from sensing of information, its determination to its presentation, and a data flow will be described.

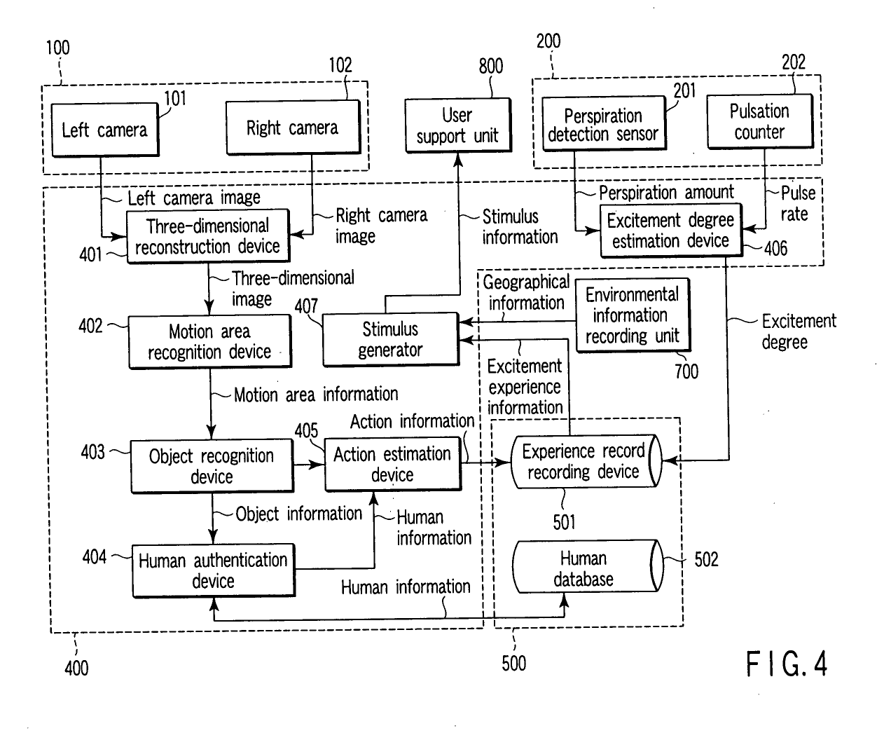

First, the process up to presentation of stimulus information to the user support unit 800 will be described by referring to FIG. 4. In this case, the user external information acquisition unit 100 includes left and right cameras 101 and 102, and the user internal information acquisition unit 200 includes a perspiration detection sensor 201 and a pulsation counter 202. The user state estimation unit 400 includes a three-dimensional reconstruction device 401, a motion area recognition device 402, an object recognition device 403, a human authentication device 404, an action estimation device 405, an excitement degree estimation device 406, and a stimulus generator 407. The user information recording unit 500 includes an experience record recording device 501, and a human database 502.

That is, left and right camera images of visual points from the user, i.e., left and right parallactic images, are obtained by the left and right cameras 101 and 102 of the user external information acquisition unit 100.

The three-dimensional reconstruction device 401 of the user state estimation unit 400 generates a three-dimensional image of the user visual point from the parallactic images sent from the left and right cameras 101 and 102. The motion area recognition device 402 recognizes a motion area in which there is a moving object from the three-dimensional image, and supplies motion area information to the object recognition device 403. The object recognition device 403 recognizes as type of the moving object, i.e., a human, an article, or the like, in the motion area, and outputs object information to the human authentication device 404, and the action estimation device 405. The human authentication device 404 refers to the human database 502 of the user information recording unit 500 to specify who the human recognized as the object is, and sends human information to the action estimation device 405. If the human has not been registered in the human database 502, it is additionally registered therein. Then, the action estimation device 405 estimates user's current action (e.g., moving or conversing) based on the object information from the object recognition device 403 and the human information from the human authentication device 404, and a result of the estimation is recorded in the experience record recording device 501 of the user information recording unit 500.

On the other hand, user's perspiration amount is obtained by the perspiration detection sensor 201 of the user internal information acquisition unit 200, and user's pulse rate is obtained by the pulsation counter 202.

The excitement degree estimation device 406 of the user state estimation unit 400 estimates how much the user is excited from the user's perspiration amount and pulse rate obtained by the perspiration detection sensor 201 and the pulsation counter 202. Then, the estimated excitement degree is recorded in the experience record recording device 501 of the user information recording unit 500.

The stimulus generator 407 of the user state estimation unit 400 generates stimulus information from the excitement experience information from the experience record recording device 501 of the user information recording unit 500 and geographical information from the environmental information recording unit 700. For example, reference is made to the environmental information recording unit 700 based on experience of a high excitement degree to generate information likely to be interesting regarding a place of a current position as stimulus information. Then, this stimulus information is transmitted to the user support unit 800 to be presented to the user.

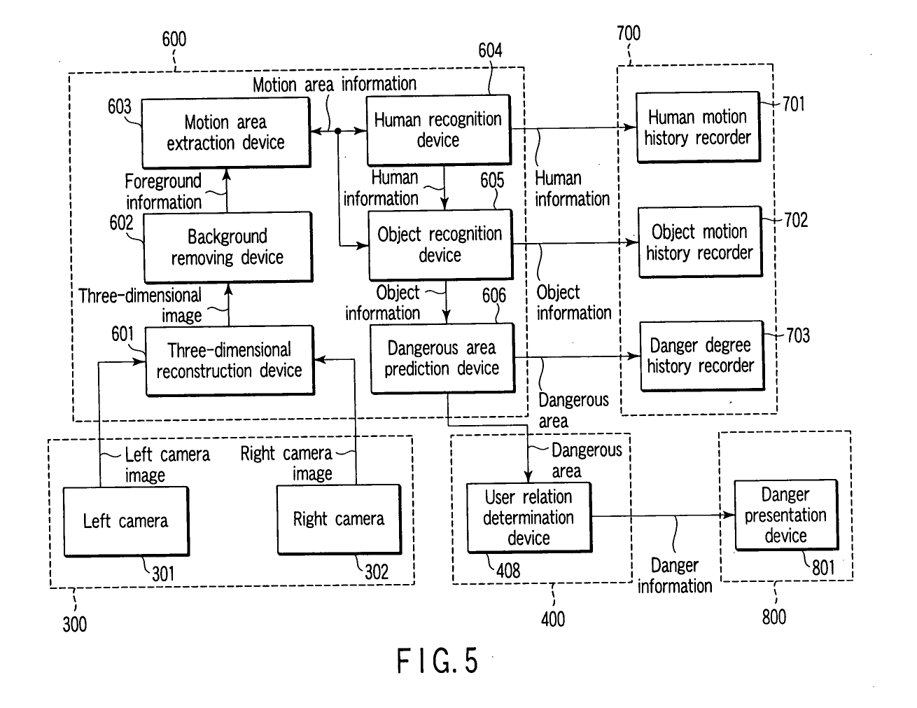

Next, the process up to presentation of danger information to the user support unit 800 will be described by referring to FIG. 5. In this case, the environmental information acquisition unit 300 includes left and right cameras 301 and 302. The environmental state estimation unit 600 includes a three-dimensional reconstruction device 601, a background removing device 602, a motion area extraction device 603, a human recognition device 604, an object recognition device 605, and a dangerous area prediction device 606. The environmental information recoding unit 700 includes a human motion history recorder 701, an object motion history recorder 702, and a danger degree history recorder 703. Additionally, in this case, the user sate estimation unit 400 includes a user relation determination device 408, and the user support unit 800 includes a danger presentation device 801.

That is, left and right camera images of visual points predetermined positions thereof, i.e., left and right parallactic images, are obtained by the left and right cameras 301 and 302 of the environmental information acquisition unit 300.

The three-dimensional reconstruction device 601 of the environmental state estimation unit 600 generates a three-dimensional image of the visual point of the predetermined position from the parallactic images sent from the left and right cameras 301 and 302. The background removing device 602 removes background information from the generated three-dimensional image, and supplies foreground information alone to the motion area extraction device 603. The motion area extraction device 603 recognizes a motion area in which there is a moving object from the foreground information, and supplies motion area information to the human recognition device 604 and the object recognition device 605. The human recognition device 604 picks up humans alone (user group) among moving objects included in the motion area to generate human information indicating a position and a moving direction of each (each user), and supplies it to the object recognition device 605. The generated human information is recorded in the human motion history recorder 701 of the environmental information recorder 700. Accordingly, movement history of each human within the visual fields of the left and right cameras 301 and 302 of the environmental information acquisition unit 300 is recorded in the human motion history recorder 701. In this case, the human recognition device 604 does not specify who each human is different from the case of the human authentication device 404 of the user state estimation unit 400. Thus, a possibility of violating privacy is small.

The object recognition device 605 recognizes, based on the human information from the human recognition device 604, a moving object other than the human among those moving in the motion area indicate by the motion area information sent from the motion area extraction device 603. Then, a position and a moving direction of each human indicated by the human information, and a position and a moving direction of each moving object are generated as object information, and supplied to the dangerous area prediction device 606. Additionally, the object information is recorded in the object motion history recorder 702 of the environment information recording unit 700. Accordingly, movement history of each human and each moving object within the visual fields of the environmental information acquisition unit 300 is recorded in the object motion history recorder 702.

The dangerous area prediction device 606 predicts an area in which there is a danger of collision between humans or between a human and a moving object after a certain time based on the object information sent from the object recognition device 605. In this case, if an interface is disposed in the dangerous area prediction device 606 to receive a danger degree attribute input from the outside, it is possible to input information regarding planned construction, a broken-down car, a disaster, a weather, an incident (crime rate, or situation induced by past incident) or the like beforehand, thereby increasing reliability of danger prediction. Then, for example, dangerous area information indicating the predicted dangerous area is transmitted to an area including at least the dangerous area by radio, and recorded in the danger degree history recorder 703 of the environmental information recording unit 700. Accordingly, history of the area determined to be the dangerous area is recorded in the danger degree history recorder 703.

The user relation determination device 408 of the user state estimation unit 400 of the user receives the dangerous area information sent from the dangerous area prediction device 606 of the environmental state estimation unit 600 to determine whether the user is in the dangerous area or not. If the user is in the area, danger information is sent to the danger presentation device 801 to inform the danger to the user.

By the aforementioned information flow, the user support is realized in the form of estimating a necessary user's or environmental state and presenting a danger degree to the user.

### Second Embodiment

Next, a second embodiment of the present invention will be described. According to the embodiment, a function for privacy protection is further added.

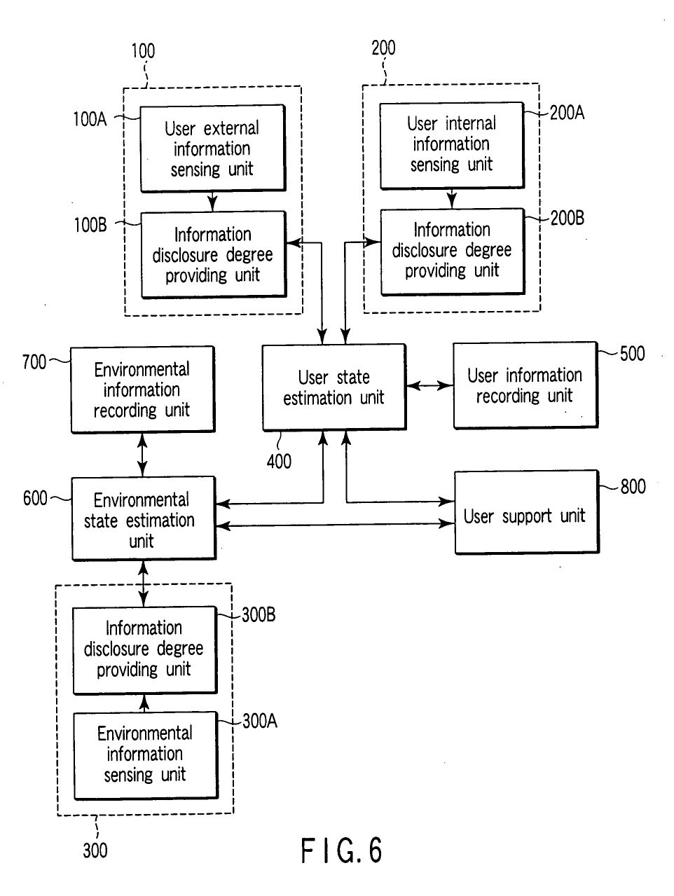

That is, as shown in FIG. 6, in the configuration of the first embodiment shown in FIG. 1, the user external information acquisition unit 100 comprises an information disclosure degree providing unit 100B for providing an information disclosure degree in addition to a user external information sensing unit 100A equivalent to the configuration of the first embodiment for obtaining the user external information. Similarly, the user internal information acquisition unit 200 comprises an information disclosure degree providing unit 200B in addition to a user internal information sensing unit 200A. The environmental information acquisition unit 300 comprises an information disclosure degree providing unit 300B in addition to an environmental information sensing unit 300A.

Here, the information disclosure degree is information indicating conditions under which the information can be disclosed. In other words, the information disclosure degree designates the system or user that disclose the user information, in order to limit the disclosure in accordance with an attribute of an information receiving side, e.g., the information may be disclosed to the user only, to family members, or to those of similar hobbies. This information disclosure degree may be changed based on information estimated by the user state estimation unit 400 of the environmental state estimation unit 600. For example, if an object is not estimated to be a human by the environmental state estimation unit 600, it is permitted to increase the number of users or systems to which the information is disclosed.

Hereinafter, by limiting functions for simplicity, a specific configuration from sensing of information, its determination to its presentation, and a data flow will be described.

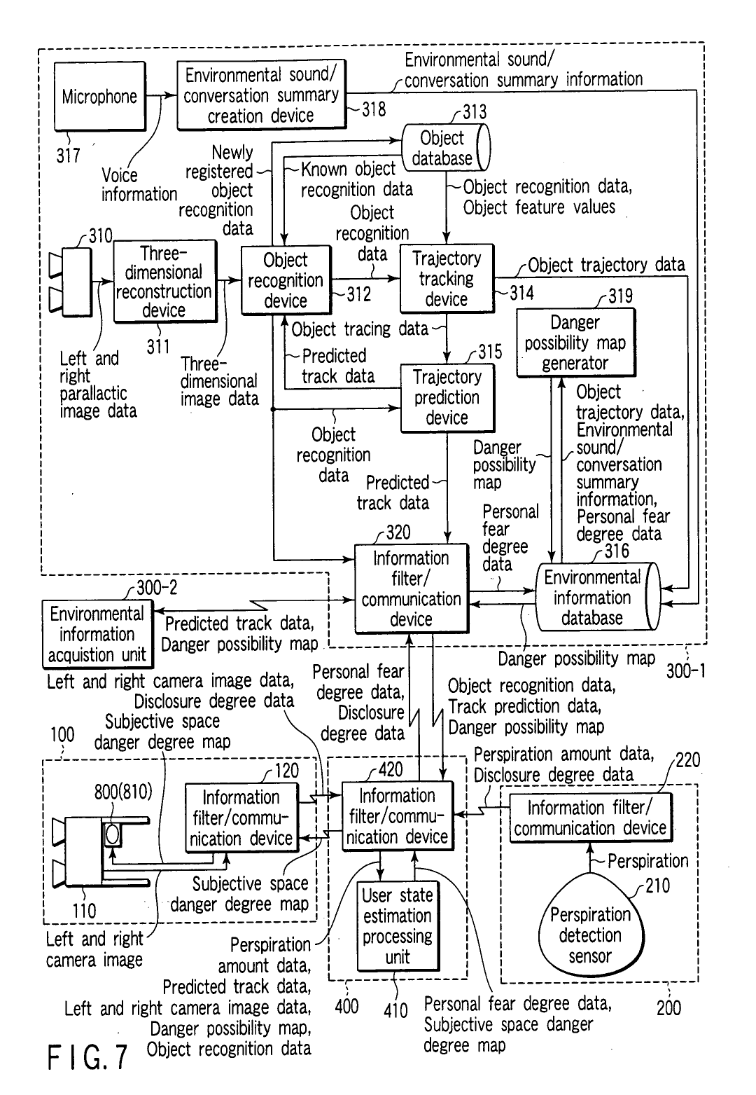

First, the process up to presentation of stimulus information to the user support unit 800 will be described by referring to FIG. 7. In this case, the user external information acquisition unit 100 includes a spectacles type stereo camera 110 as the user external information sensing unit 100A, and an information filter/communication device 120 as the information disclosure degree providing unit 100B. A microdisplay 810 is mounted as the user support unit 800 to the spectacles type stereo camera 110. The user internal information acquisition unit 200 includes a perspiration detection sensor 210 as the user internal information sensing unit 200A, and an information filter/communication device 220 as the information disclosure degree providing unit 200B. The user state estimation unit 400 includes a user state estimation processing unit 410, and an information filter/communication device 420. Additionally, in this case, there are a plurality of environmental information acquisition units 300 (300-1, 300-2). One environmental information acquisition unit 300-1 includes a stereo camera 310, a three-dimensional reconstruction device 311, an object recognition device 312, an object database 313, a trajectory tracking device 314, a trajectory prediction device 315, an environmental information database 316, a microphone 317, an environmental sound/conversation summary creation device 318, a danger possibility map generator 319 as the environmental information sensing unit 300A, and an information filter/communication device 320 as the information disclosure degree providing unit 300B.

Here, the spectacles type stereo camera 110 of the user external information acquisition unit 100 obtains left and right camera images as left and right parallactic images of user visual points. The information filter/communication device 120 transmits data of the left and right camera images together with disclosure degree data to the user state estimation unit 400.

The perspiration detection sensor 210 of the user internal information acquisition unit 200 obtains user's perspiration amount. The information filter/communication device 220 transmits the obtained perspiration amount data together with its disclosure degree data to the user state estimation unit 400.

The information filter/communication device 420 of the user state estimation unit 400 receives pieces of information sent from the user external information acquisition unit 100, the user internal information acquisition unit 200, and the environmental information acquisition units 300-1, 300-2, and filters the received data based on each disclosure degree data of the received data and supplies the filtered data to the user state estimation processing unit 410. This user state estimation processing unit 410 estimates various user states by using the supplied data. For example, a level of user's fear is determined based on the perspiration amount data from the user internal information acquisition unit 200, and its result can be transmitted as personal fear degree data together with the disclosure degree data to the information filter/communication device 420.

The stereo camera 310 of the environmental information acquisition unit 300-1 is installed in a predetermined place, and configured to obtain left and right parallactic image data of its predetermined position visual point and to send the data to the three-dimensional reconstruction device 311. This three-dimensional reconstruction device 311 generates three-dimensional image data of the predetermined position visual point from the parallactic image data sent from the stereo camera 310, and sends the data to the object recognition device 312. The object recognition device 312 recognizes a moving object from the three-dimensional image data generated by the three-dimensional reconstruction device 311 by referring to known object data registered in the object database 313, and transmits the object recognition data to the trajectory tracking device 314, the trajectory prediction device 315, and the information filter/communication device 320. A function is also provided to register the object recognition data as newly registered object recognition data in the object database 313. It is to be noted that when executing object recognition, the object recognition device 312 can increase recognition accuracy by using predicted track data from the track prediction device 315.

The trajectory tracking device 314 calculates a moving track, a speed, a size or the like of each object based on past object recognition data and feature values of the object registered in the object database 313. Its result is supplied as object trajectory data to the trajectory prediction device 315, and registered in the environmental information database 316. The trajectory prediction device 315 estimates a future position or speed of each object from the object tracing data from the trajectory tracking device 314 and the object recognition data from the object recognition device 312. Then, a result of the estimation is supplied as predicted track data to the object recognition device 312 and the information filter/communication device 320.

On the other hand, the microphone 317 obtains voice information. The environmental sound/conversation summary creation device 318 comprises a function of separating an environmental sound from the voice information, voice-recognizing conversation contents to create a summary, and registering the summary as environmental sound/conversation summary information in the environmental information database 316.

The personal fear data from the user state estimation unit 400 which has been received by the information filter/communication device 320 and filtered in accordance with the disclosure degree data is also registered in the environmental information database 316. The danger possibility map generator 319 creates a danger possibility map indicating an area in which there is a possibility of danger from the tracing data, the environmental sound/conversation summary information, and the personal fear data registered in the environmental information database 316, and registers the map in the environmental information database 316.

The information filter/communication device 320 comprises a function of exchanging the predicted track data from the trajectory prediction device 315 and the personal fear data registered in the environmental information database 316 with the other environmental information acquisition unit 300-2, and a function of transmitting the predicted track data, the personal fear data, and the object recognition data recognized by the object recognition device 312 to the user state estimation unit 400 of each user. In the environmental information acquisition units 300-1 and the 300-2, no data is generated to specify an individual. Thus, even if disclosure degree data is added during transmission, there is only a small possibility of violating privacy. However, preferably, the data are transmitted together with the disclosure degree data.

The user state estimation processing unit 410 of the user state estimation unit 400 includes a function of generating the personal fear data. Further, the user state estimation processing unit 410 determines a danger degree of the user based on the predicted track data, the personal far data and the object recognition data from the environmental information acquisition units 300-1 and 300-2, and the left and right camera image data from the user external information acquisition unit 100 to generate a subjective space danger degree map. Then, the map is transmitted through the information filter/communication device 420 to the user support unit 800 in which a disclosure degree has been set. Accordingly, for example, it is possible to inform the danger to the user by displaying the subjective space danger degree map on the microdisplay 810 mounted to the spectacles type stereo camera 110 of the user external information acquisition unit 100.

In the foregoing, the object recognition data and the predicted track data generated by the object recognition device 312 and the trajectory prediction device 315 are transmitted from the environmental information acquisition unit 300-1. However, these data may be first registered in the database, and then transmitted. Nonetheless, in view of emergency, such transmission of real time data is preferable.

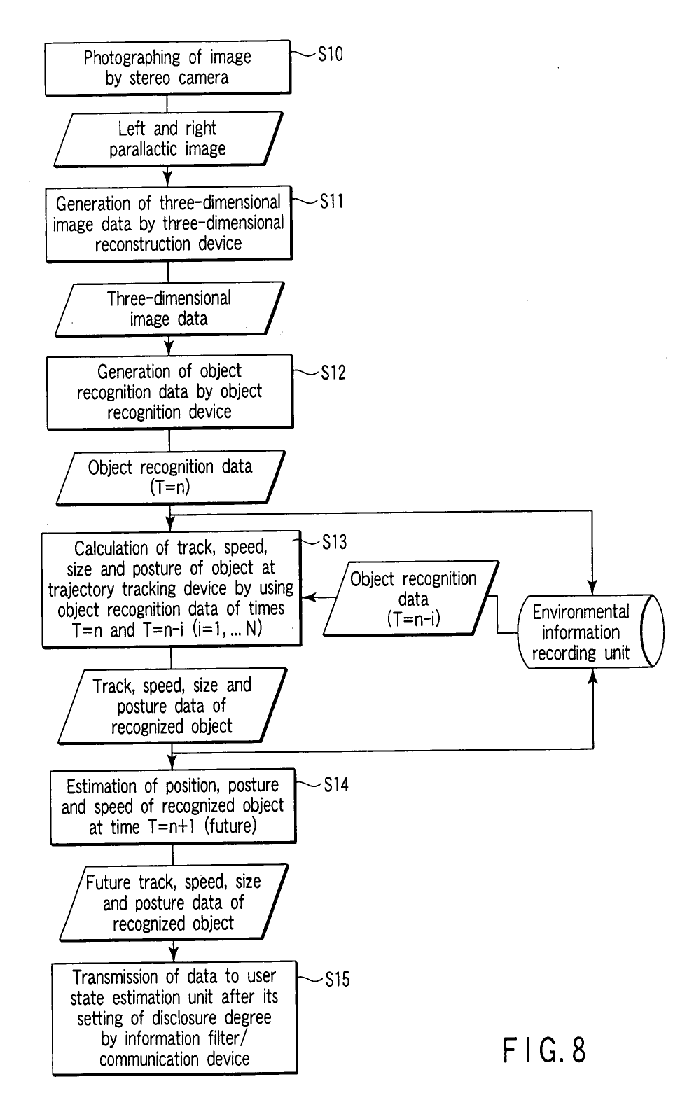

Now, an operation of generating predicted track data and then transmitting information to the user state estimation unit 400 in the environmental information acquisition unit 300-1 will be described by referring to a flowchart of FIG. 8.

That is, an image is photographed by the stereo camera 310 (step S10) to obtain a left and right parallactic image. Next, three-dimensional data is generated from the left and right parallactic image by the three-dimensional reconstruction device 311 (step S11). Then, object recognition data at a time T=n is generated from the three-dimensional data by the object recognition device 312 (step S12). This object recognition data is registered in the object database 313 as the environmental information recording unit.

Next, at the trajectory tracking device 314, object tracing data of the recognized object, i.e., a track, a speed, a sized and posture information of the object, is calculated by using the object recognition data of the time T=n from the object recognition device 312, object recognition data at times T=n−i (i=1, . . . N) registered in the object database 313 as the environment information recording unit, and feature values of the recognized object registered in the object database 313 (step S13). The calculated object tracking data is registered in the environmental database 316 as the environmental information recording unit.

Subsequently, at the trajectory prediction device 315, a position, posture information and a speed of the recognized object at a time T=n+1, i.e., in the future, are estimated (step S14). Then, after a disclosure degree is set by the information filter/communication device 320, the estimated data is transmitted as predicted tracking data together with other data (object recognition data and danger possibility map) to the user state estimation unit 400 (step S15).

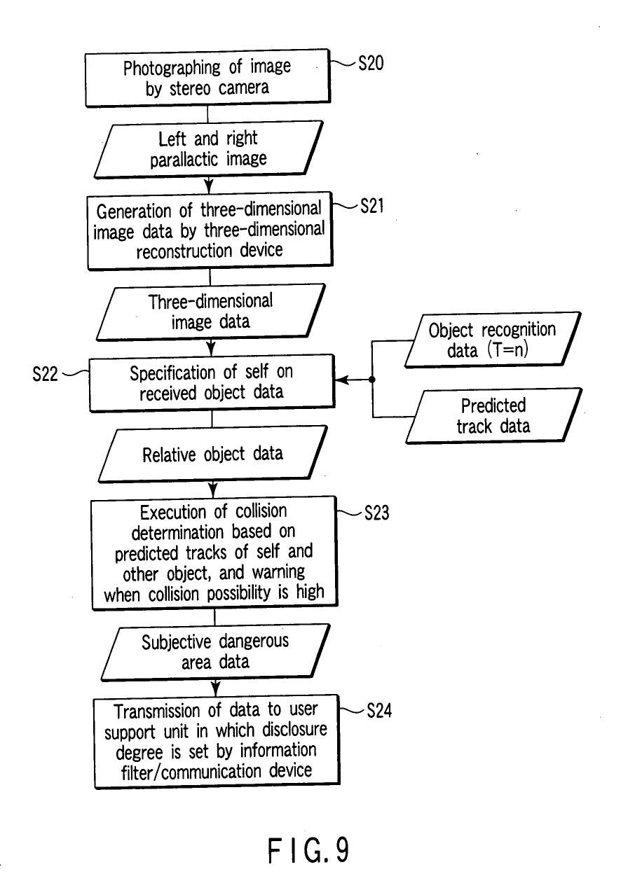

Next, an operation of the user state estimation unit 400 of generating the subjective space danger data from the left and right camera image data from the user external information acquisition unit 100 and the object recognition data and the predicted tracking data from the environmental information acquisition unit 300-1 will be described by referring to a flowchart of FIG. 9.

That is, an image is captured by the stereo camera 110 of the user external information acquisition unit 100 to obtain a left and right parallactic image (step S20). The three-dimensional reconstruction device (not shown) in the user state estimation processing unit 410 receives the left and right parallactic image to generate three-dimensional image data (step S21). Next, at the user state estimation processing unit 410 detects own position and/or posture information from the three-dimensional image data, and specifies the user himself/herself from the object data sent from the environmental information acquisition unit 300-1, i.e., the object recognition data of the time T=n and the predicted tracking data (step S22) to obtain relative object data.

Next, collision determination is made based on the predicted track data of the user himself and the other object (recognized object), and subjective dangerous area data is generated for warning when a collision possibility is high (step S23). Then, the subjective dangerous area data is transmitted to the user support unit 800 in which the disclosure degree has been set by the information filter/communication device 420, e.g., the microdisplay 810 mounted to the spectacle type stereo camera 110 which the user wears (step S24).

For example, when a dubious object approaches from behind, information on the dubious object such as identification of the dubious object, a car or a human, a drowsy state of a driver if the object is a car, or looking sideways, is obtained from the user state estimation unit 400 of dubious object's own within a range of protecting minimum privacy based on an information disclosure degree added to each information, whereby danger can be prevented more accurately. For the information disclosure degree, for example, if there are a name, an age, a height, weight, a physical condition, an action state, and an excitement degree as personal information, by disclosing the height, the physical condition and the action state alone to all, only the three pieces of information are disclosed while the others are not. For example, the user who detects drowsing can know a possibility of an accident beforehand.

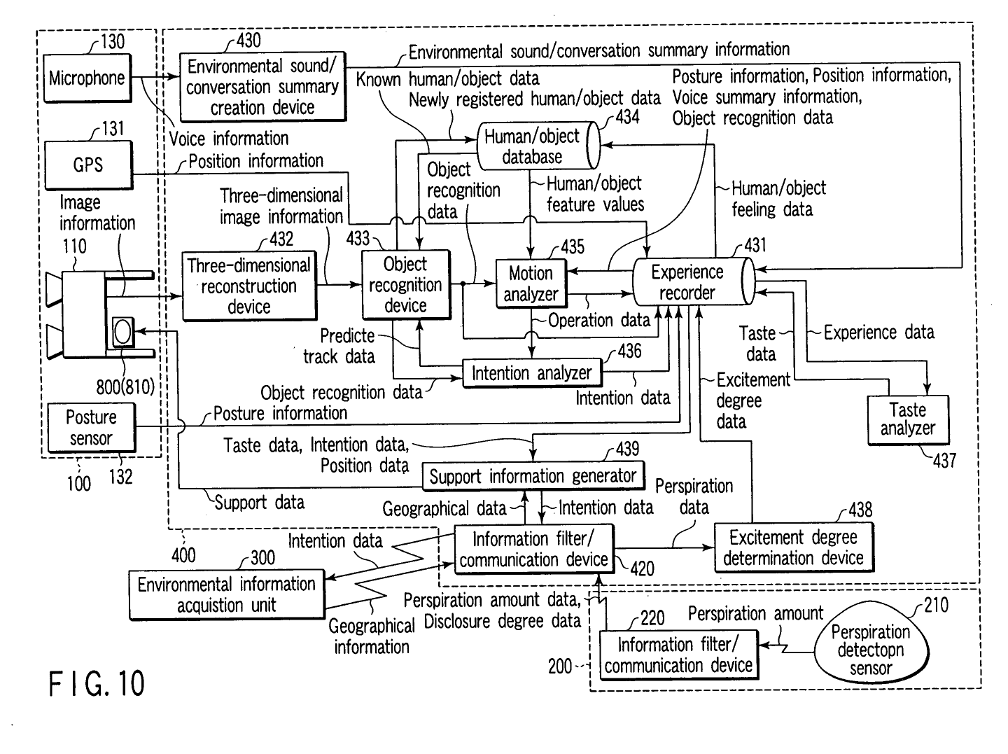

Next, the process up to presentation of information based on user's taste and preference to the user support unit 800 will be described by referring to FIG. 10. In this case, the user external information acquisition unit 100 includes an eyeglass type stereo camera 110, a microphone 130, and a GPS sensor 131, and a posture sensor 132 as the user external information sensing unit 10A. A microdisplay 810 is mounted as the user support unit 800 to the spectacles stereo camera 110. The user internal information acquisition unit 200 includes a perspiration amount sensor 210 as the user internal information sensing unit 200A, and an information filter/communication device 220 as the information disclosure degree providing unit 200B. The user state estimation unit 400 includes, as the user state estimation processing unit 410, an environmental sound/conversation summary creation device 430, an experience recorder 431, a three-dimensional reconstruction device 432, an object recognition device 433, a human/object database 434, a motion analyzer 435, an intention analyzer 436, a taste analyzer 437, an excitement degree determination device 438, and a support information generator 439, and an information filter/communication device 420.

Here, the microphone 130 of the user external information acquisition unit 100 obtains voice information of a range within which the user can sense objects. The voice information obtained by the microphone 130 is transmitted to the environmental sound/conversation summary creation device 430 of the user state estimation unit 400. This environmental sound/conversation summary estimation unit 430 comprises a function of separating the environmental sound from the voice information, voice-recognizes conversation contents to create a summary, and registering it as environmental sound/conversation summary information in the experience recorder 431.

The GPS sensor 131 of the user external information acquisition unit 100 receives a GPS signal from a satellite to obtain position information indicating user's position. This position information is transmitted to the experience recorder 431 of the user state estimation unit 400 to be registered therein. The posture sensor 132 of the user external information acquisition unit 100 obtains posture information indicating user's direction. This posture information is similarly transmitted to the experience recorder 431 of the user state estimation unit 400 to be registered therein.

The eyeglass-type stereo camera 110 of the user external information acquisition unit 100 obtains left and right camera images as left and right parallactic images of user visual points. The left and right camera image data is transmitted to the three-dimensional reconstruction device 432 of the user state estimation unit 400. The three-dimensional reconstruction device 432 generates three-dimensional image data of user's visual point from the received image data, and sends the data to the object recognition device 433. The object recognition device 433 recognizes humans and objects from the three-dimensional data generated by the three-dimensional reconstruction device 432 by referring to the known human/object data registered in the human/object database 434. Then, a result of the object recognition is transmitted as object recognition data to the motion analyzer 435 and the intention analyzer 436, and registered in the experience recorder 431. A function is also provided to register, if there is a human or an object not registered in the human/object database 434, object recognition data thereof as newly registered human/object recognition data in the human/object database 434. It is to be noted that when executing object recognition, the object recognition device 433 can increase recognition accuracy by using intention data from the intention analyzer 436.

The motion analyzer 435 of the user state estimation unit 400 analyzes posture information, a position, and user's motion based on the object recognition data from the object recognition device 433 and on the posture information, the position information, the voice summary (environmental sound/conversation abstract) information, and past object recognition data registered in the experience recorder 431. Then, its result is supplied as motion data to the intention analyzer 436, and registered in the experience recorder 431. The intention analyzer 436 analyses user's action intention from the motion data from the motion analyzer 435 and the object recognition data from the object recognition device 433. Then, a result of the analysis is supplied as intention data to the object recognition device 433, and registered in the experience recorder 431.

The perspiration detection sensor 210 of the user internal information acquisition unit 200 obtains user's perspiration amount. The information filter/communication device 220 transmits the obtained perspiration amount data together with disclosure degree data to the user state estimation unit 400.

The information filter/communication device 420 of the user state estimation unit 400 comprises a function of receiving the information sent from the user internal information acquisition unit 200, and filtering the received data in accordance with disclosure degree data. The excitement degree determination device 438 determines how much the user is excited based on the perspiration amount data of the user internal information acquisition unit 200 filtered by the information filter/communication device 420, and registers a result thereof as excitement degree data in the experience recorder 431. The taste analyzer 437 analyzes user's taste from various experience data registered in the experience recorder 431, and registers a result of the analysis as taste data in the experience recorder 431.

For example, for a customer situation of a restaurant currently destined for, information of a high disclosure degree but unrelated to privacy such as with children, conversing among housewife friends, or excitedly talking loudly is obtained from customer's user state estimation unit. Accordingly, it is possible to check whether the situation satisfiers user's current frame of mind, e.g., “wish to dine quietly” while maintaining privacy. Additionally, a situation in the restaurant may be directly sent in a form of a moving image. In this case, however, user's face whose disclosure degree is limited for privacy protection is distributed in mosaic or the like.

The support information generator 439 of the user state estimation unit 400 generates support data by referring to necessary information from the environmental information acquisition unit 300 based on the taste data, the intention data, the position data and the like registered in the experience recorder 431. Then, for example, the generated support data is presented to the microdisplay 810 mounted as the user support unit 800 to the eyeglass-type stereo camera 110 of the user external information acquisition unit 100. For example, the intention data is transmitted through the information filter/communication device 420 to the environmental information acquisition unit 300, geographical information related to the intention data is similarly received through the information filter/communication device 420 from the environmental information acquisition unit 300, and the geographic data is displayed as support data on the microdisplay 810. It is to be noted that disclosure degrees may be set during the transmission/reception of the intention data and the geographic data.

Now, the operation of generating the support data in the user state estimation unit 400 will be described by referring to a flowchart of FIG. 11.

That is, an image is captured by the stereo camera 110 of the user external information acquisition unit 100 to obtain a left and right parallactic image (step S30). The three-dimensional reconstruction device 432 receives the image to generate three-dimensional image data (step S31). This three-dimensional image data is registered in the experience recorder 431. Then, at the object recognition device 433, object recognition data at a time T=n is generated from the three-dimensional data (step S32). This object recognition data is also registered in the experience recorder 431.

On the other hand, body posture information of the user is detected by the posture sensor 132 of the user external information acquisition unit 100 (step S33), and registered as posture information of the time T=n in the experience recorder 431. Similarly, a GPS signal is received by the GPS sensor 131 of the user external information acquisition unit 100 (step S34), and registered as position information at the time T=n in the experience recorder 431. Additionally, a voice is recorded by using the microphone 130 of the user external information acquisition unit 100 (step S35), and voice information is sent to the environmental sound/conversation creation device 430. Then, a summary is created by the environmental sound/conversation summary creation device 430 (step S36), and registered as voice summary information of the time T=n in the experience recorder 431.

Next, at the motion analyzer 435, posture information, a position, and user's motion are analyzed by using the object recognition data of the time T=n from the object recognition device 433, and the object recognition data at times T=n−i (I=1, . . . N), the posture information, the position information, and the voice summary information registered in the experience recorder 431 (step S37). From operation data of the time T=n which is a result of the analysis, user's action intention is analyzed at the intention analyzer 436 (step S38). Intention data of the result is registered in the experience recorder 431.

Then, the support information generator 439 generates support data by referring to necessary information from the user's action intention indicated by the intention data and the user's taste indicated by the taste data registered in the experience recorder 431, e.g., the map information, from the environmental information acquisition unit 300 (step S39). This support data is presented on the microdisplay 810 to the user (step S40).

### Third Embodiment

Next, a specific information presentation example in the user support apparatus of the first and second embodiments will be described as a third embodiment of the present invention.

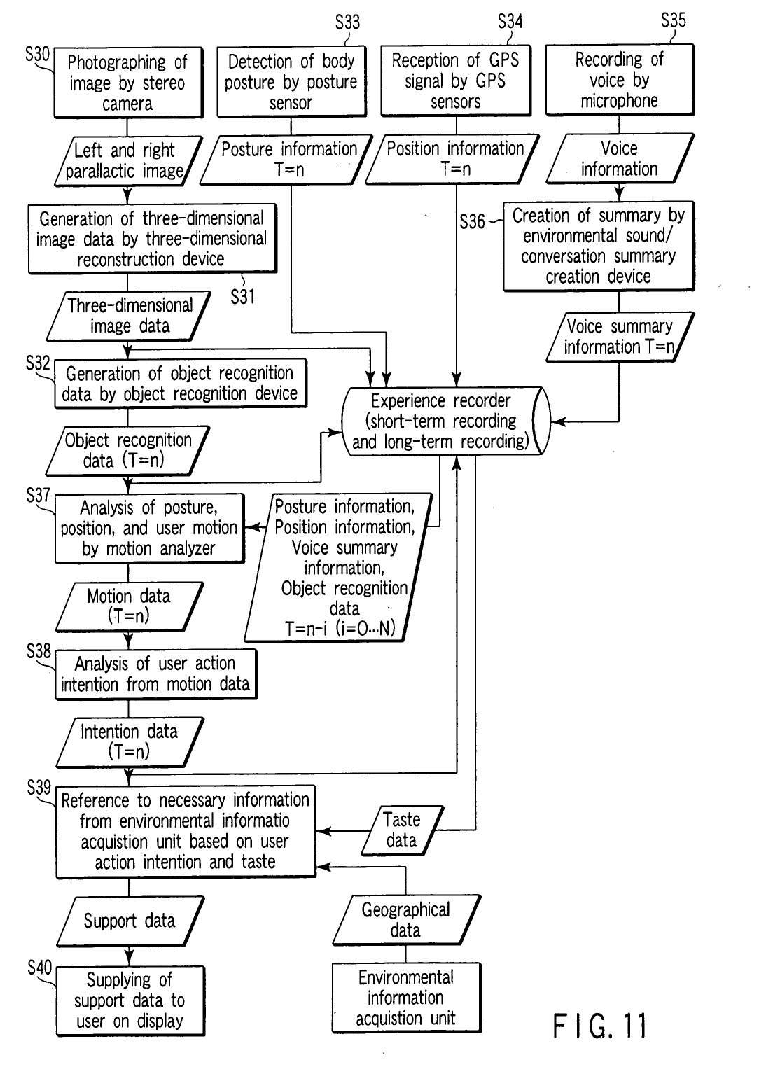

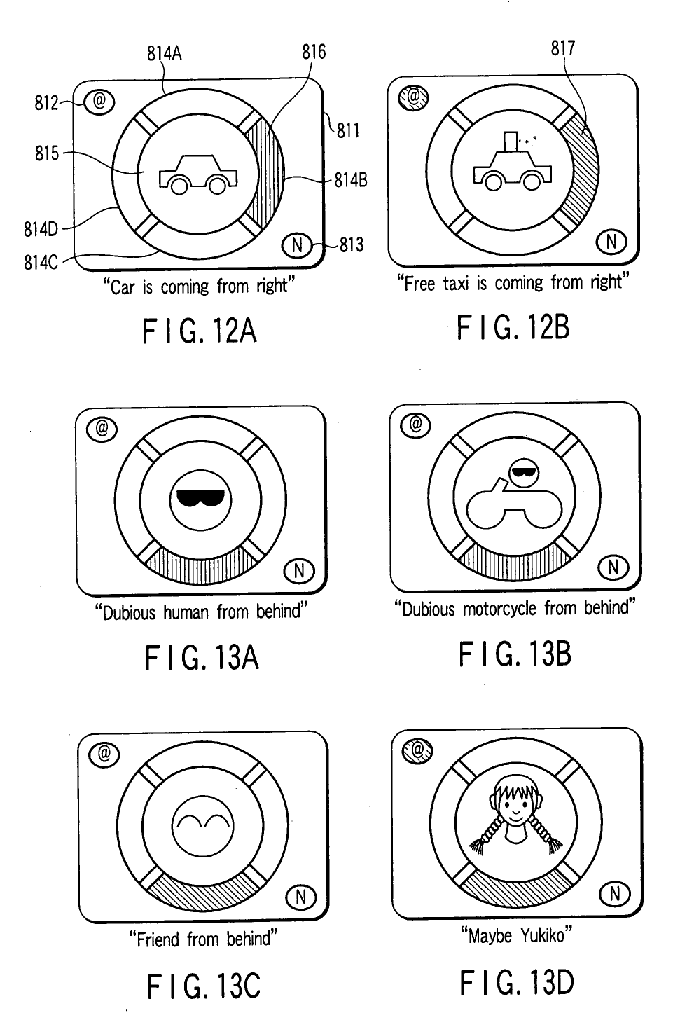

For example, the microdisplay 810 as the user support unit 800 included in the eyeglass-type stereo camera 110 shown in FIGS. 2A and 2B can be configured as a screen 811 shown in FIG. 12A. Here, on the screen 811, an @ mark 812, an N mark 813, upper-lower and left-right segments 814A to 814D, and a contents display unit 815 surrounded with the segments are displayed. The upper-lower and left-right segments 814A to 814D indicate user's front, back, left and right, and lit to indicate which direction of the user an object or a state as an origin of information displayed on the contents display unit 815 is present in. In this case, by changing displayed colors of the segments 814A to 814D, e.g., a red display 816 as information to alert the user, and a green display as friendly information, kinds of information presented on the contents display unit 81 can be indicated.

Simultaneously with the information presentation on the contents display unit 815, voice information regarding displayed information contents, e.g., “Vcar is coming from right”, is output through a speaker 820 as the user support unit 800 included in the spectacles type stereo camera 110, making it possible to present information. It is to be noted that the approaching of the car is estimated by the user state estimation unit 400 based on the information obtained from the user external information acquisition unit 100 or the environmental information acquisition unit 300. Moreover, by identifying a collision possibility as described above in the scene 5 of the first embodiment, a segment display can be set as a red display 816 or a green display 817 shown in FIGS. 12A and 12B.

The @ mark 812 and the N mark 813 are interfaces. For example, if the user fixers his visual line there for three sec., “yes” can be selected in the case of the @ mark 813, and “no” can be selected in the case of the N mark 813. This 3-second visual line fixing can be determined by disposing a visual line detection sensor as the user internal information acquisition unit 200, and estimating whether the user views the @ mark 812 or the N mark 813 for three sec., or more at the user state estimation unit 400. The user state estimation unit 400 displays a result of the determination. That is, FIG. 12B shows an example of presenting information when a free taxi approaches from a direction (right direction) indicated by the green display 817. The @ mark 812 is lit and displayed in green color because the user views the @ mark 812 for three seconds, or more in accordance with the presented information. In other words, the user displays intention of “yes” with respect to the presented information, and the user state estimation unit 400 can know that the presentation of information has been useful for the user. Thus, the user state estimation unit 400 can identify whether the information presented to the user has been useful or not for the user. By storing the result in the user information recording unit 500, it is possible to reduce a possibility of presenting unnecessary information to the user.

Each of FIGS. 13A and 13B shows an information presentation example when the user state estimation unit 400 estimates from the information obtained from the user external information acquisition unit 100 or the environmental information acquisition unit 300 that an unknown human or motorcycle follows user's back for a predetermined time or distance. On the other hand, if a record of human has been recorded in the user information recording unit 500, information is presented as shown in FIGS. 13C and 13D. Needless to say, if the stored record is determined to be a human of a high degree of fear, i.e., a human of bad impression based on the information obtained by the user internal information acquisition unit 200, the segments 814A to 814D are set as red displays 816.

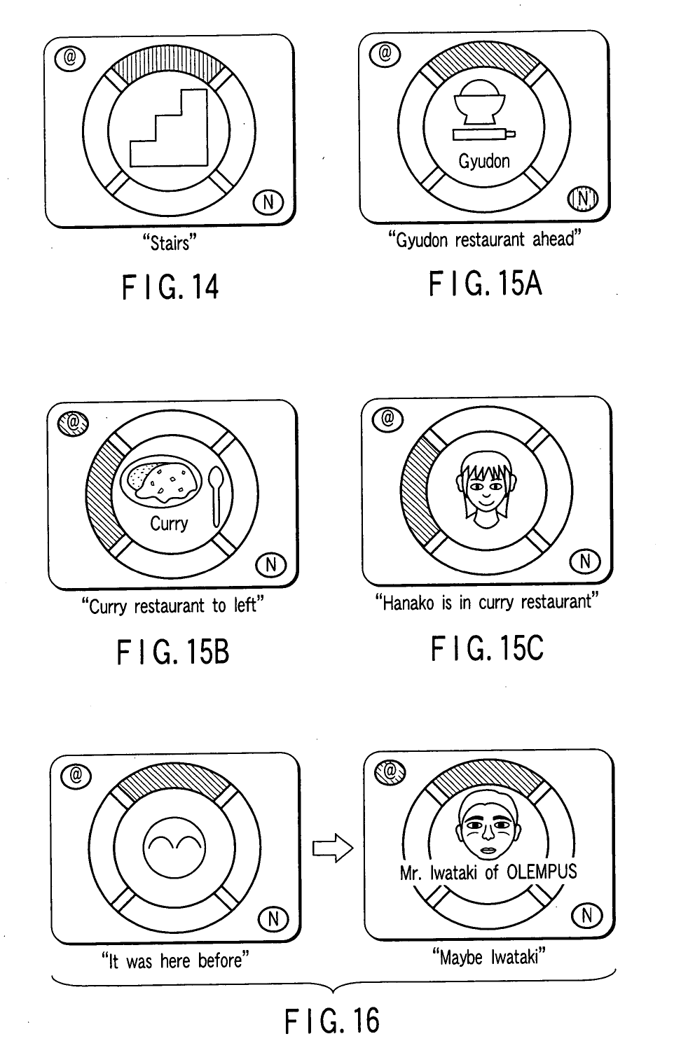

As shown in FIG. 14, the user state estimation unit 400 can present not only a moving object but also information to alert the user based on the information obtained from the user external information acquisition unit 100 or the environmental information acquisition unit 300.

In the case of the scene 2 of the first embodiment, information presentation is as shown in FIG. 15A or 15B. Additionally, in this case, for example, information can be presented as shown in FIG. 15C by identifying user's acquaintance among people in a curry restaurant based on the information from the environmental information acquisition unit 300 installed in the curry restaurant.

Furthermore, for the known human, information can be more effectively presented by not employing any one of the information presentations of FIGS. 13C and 13D but changing presented information contents as shown in FIG. 16.

The present invention has been described on the basis of embodiments. Needless to say, however, the embodiments are in now way of the invention, and various modifications and applications can be made within the main teaching of the invention. For example, according to the embodiments, the human is the user. However, the user may be a robot rather than a human, and any moving object can be employed such as an automobile and a train.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A user support apparatus comprising:
a user information acquisition unit configured to obtain information regarding a user;
a user state estimation unit configured to estimate at least one of user's position information, posture information, physical sate, and mental state as a user state based on the information obtained by the user information acquisition unit; and
a user support unit configured to support at least one of user's action, memory and thinking based on the user state estimated by the user state estimation unit.

2. The apparatus according to claim 1, wherein the user information acquisition unit includes an information disclosure degree adding unit configured to add an information disclosure degree as an attribute to designate at least one of the user and a system that disclose the user information.

3. A user support apparatus comprising:
a user sate estimation unit including at least two of a user external information acquisition unit configured to obtain user external information which is information sensed by a user, a user internal information acquisition unit configured to obtain user internal information which is user's own information, and an environmental information acquisition unit configured to obtain environmental information around the user, and configured to estimate a user state containing at least one of user's position information, posture information, physical state and mental state based on the pieces of information obtained by the acquisition units; and
a user support unit configured to support at least one of user's action, memory and thinking based on the user state estimated by the user state estimation unit.

4. The apparatus according to claim 3, wherein at least one of the user external information acquisition unit and the user internal information acquisition unit is mounted to the user.

5. The apparatus according to claim 3, wherein the user external information acquisition unit supports a range in which the user can feel by some of five senses, and obtains at least one of an image, a voice, an odor, an air temperature, a humidity, components in air, an atmospheric pressure, brightness, an ultraviolet ray amount, an electric wave, a magnetic field, a ground temperature, IC tag information, a distance, and a wind direction.

6. The apparatus according to claim 3, wherein user external information acquisition unit obtains at least one of user's position information and user's posture information by using at least one of a one-dimensional range sensor, a two-dimensional range sensor, a gravitational direction sensor, an accelerometer sensor, an angular accelerometer sensor, a gyrosensor, position and posture estimation information based on sensing of a maker fixed to the outside, and a GPS signal.

7. The apparatus according to claim 3, wherein the user external information acquisition unit includes an interface configured to receive an input from the user.

8. The apparatus according to claim 3, wherein the user external information acquisition unit includes an information disclosure degree adding unit configured to add a disclosure range of information as an attribute to the information to be communicated.

9. The apparatus according to claim 3, wherein the user internal information acquisition unit obtains at least one of user's perspiration amount, skin potential response and level, eye movement, electromyogram, electroencephologram, brain magnetic field, vial sign, facial expression, face color, voiceprint, shaking motion, body movement, blood sugar, body fat, blood flow, saliva components, breath components, excrement components, and sweat component information.

10. The apparatus according to claim 3, wherein the user internal information acquisition unit includes an information disclosure degree adding unit configured to add a disclosure range of information as an attribute to the communication to be communicated.

11. The apparatus according to claim 3, wherein the environmental information acquisition unit includes an environment sensing unit configured to support one of a range in which a user group including the user acts and a range likely to affect the user group, and to obtain at least one of an image, a sound, an odor, an air temperature, a humidity, components in air, an atmospheric pressure, brightness, an ultraviolet ray amount, an electric wave, a magnetic field, a ground temperature, IC tag information, a distance, and a wind direction.

12. The apparatus according to claim 3, wherein the environmental information acquisition unit obtains at least one of position information and posture information of the environmental information acquisition unit itself by using at least a one-dimensional range sensor, a two-dimensional range sensor, a gravitational direction sensor, an accelerometer, an angular accelerometer, a gyrosensor, and a GPS signal.

13. The apparatus according to claim 3, further comprising an environmental state estimation unit configured to recognize at least one of history of the use group, a movement of an object group, an operation record of at least one of a vehicle, an elevator, a gate and a door, and weather from the information obtained by the environmental information acquisition unit and to generate environmental state information.

14. The apparatus according to claim 13, wherein the environmental state estimation unit includes a danger degree attribute setting unit configured to set a danger degree attribute in a virtual space based on at least one of the pieces of information obtained by the environmental information acquisition unit.

15. The apparatus according to claim 14, wherein the danger degree attribute setting unit detects a moving object around the user based on at least one of the pieces of information obtained by the environmental information acquisition unit, and predicts a danger degree attribute after a fixed time.

16. The apparatus according to claim 14, wherein the danger degree attribute setting unit includes an interface which receives a danger degree attribute input from the outside.

17. The apparatus according to claim 3, further comprising an environmental information recording unit configured to record the environmental information obtained by the environmental information acquisition unit.

18. The apparatus according to claim 17, wherein the environmental information recording unit weights newly recording information based on at least one of at least one of the user external information, the user internal information, and the environmental information, and past information recorded in the environmental information recording unit.

19. The apparatus according to claim 18, wherein the environmental information recording unit performs one of deletion and compression of information based on the weighting.

20. The apparatus according to claim 13, further comprising an environmental information recording unit configured to record the environmental state information obtained by the environmental state estimation unit.

21. The apparatus according to claim 20, wherein the environmental information recording unit weights newly recording information based on at least one of at least one of the user external information, the user internal information, and the environmental information, and past information recorded in the environmental information recording unit.

22. The apparatus according to claim 21, wherein the environmental information recording unit performs one of deletion and compression of information based on the weighting.

23. The apparatus according to claim 3, wherein the user state estimation unit obtains an interest area based on at least one of the user external information, the user internal information, and the environmental information.

24. The apparatus according to claim 3, wherein the user state estimation unit includes a user experience information extracting unit configured to extract at least one of user's position information, posture information, a point of attention, conversation contents, conversation opponent, face color of the conversation opponent, voice tone, and a visual line as user experience information based on at least one of the user external information, the user internal information and the environmental information.

25. The apparatus according to claim 24, wherein the user state estimation unit recognizes at least one of user's degree of stress, degree of excitement, degree of impression, degree of fatigue, degree of attention, degree of fear, degree of concentration, degree of attention, degree of jubilation, degree of drowsiness, degree of excretion wish, and degree of appetite by using at least one of the user external information, the user internal information, the environmental information, and the user experience information extracted by the user experience information extracting unit.

26. The apparatus according to claim 24, wherein the user state estimation unit estimates user's current action by using at least one of the user external information, the user internal information, the environmental information, and the user experience information extracted by the user experience information extracting unit.

27. The apparatus according to claim 3, further comprising a user information recording unit configured to record the user external information, the user internal information, the environmental information, and the information obtained by the user state estimation unit.

28. The apparatus according to claim 27, wherein the user information recording unit weights newly recorded information based on at least one of at least one of the user external information, the user internal information, the environmental information, and the user experience information, and past information recorded in the user information recording unit.

29. The apparatus according to claim 28, wherein the user information recording unit performs one of deletion and compression of the information based on the weighting.

30. The apparatus according to claim 3, further comprising an information presenting unit configured to present information to the user.

31. The apparatus according to claim 30, wherein the information presenting unit is mounted to the user external information acquisition unit.

32. The apparatus according to claim 30, wherein the information presenting unit is mounted to the user internal information acquisition unit.

33. The apparatus according to claim 30, wherein the information presenting unit is mounted to the environmental information acquisition unit.

34. The apparatus according to claim 30, wherein:
at least one of the user internal information acquisition unit and the user external information acquisition unit includes an information disclosure degree adding unit configured to add an information disclosure degree indicating a disclosure range of information to the information to be communicated, and
the information presenting unit selects information to be presented based on the information disclosure degree added by the information disclosure degree adding unit.

35. The apparatus according to claim 30, wherein the information presenting unit presents the information by using at least one of a sound, an image, an odor, a tactile sense, and vibration.

36. A user support apparatus comprising:
user information obtaining means for obtaining information regarding a user;
user state estimation means for estimating at least one of user's position information, posture information, physical sate, and mental state as a user state based on the information obtained by the user information obtaining means; and
user support means for supporting at least one of user's action, memory and thinking based on the user state estimated by the user state estimation means.

37. A user support apparatus comprising:
user sate estimation means including at least two of user external information acquisition means for obtaining user external information which is information sensed by a user, user internal information acquisition means for obtaining user internal information which is user's own information, and environmental information acquisition means for obtaining environmental information around the user, and for estimating a user state containing at least one of user's position information, posture information, physical state and mental state based on the pieces of information obtained by the acquisition means; and
user support means for supporting at least one of user's action, memory and thinking based on the user state estimated by the user state estimation means.
